(19) United States
(12) Patent Application Publication
(10) Pub. No.: US 2026/0126439 A1
(43) Pub. Date: May 7, 2026

(54) METHODS AND APPARATUS FOR DETERMINING THE AMOUNT OF AN ANALYTE IN A FLUID USING A PERIODIC WAVEFORM

(71) Applicant: NUTROMICS TECHNOLOGY PTY LTD, BRUNSWICK, VICTORIA (AU)

(72) Inventors: ALASTAIR HODGES, BRUNSWICK, VICTORIA (AU); STANLEY TEO, BRUNSWICK, VICTORIA (AU); JIEZHEN LI, BRUNSWICK, VICTORIA (AU)

(21) Appl. No.: 19/118,983

(22) PCT Filed: Aug. 2, 2023
(86) PCT No.: PCT/AU2023/050722

§ 371 Date: Apr. 7, 2025

Related U.S. Application Data
(60) Provisional application No. 63/414,975, filed on Oct. 11, 2022.

(51) Int. Cl. G01N 33/543; G01N 27/327; G01N 27/48; G01N 33/53

(52) U.S. Cl. CPC G01N 33/5438; G01N 27/3276; G01N 27/3277; G01N 27/48; G01N 33/5308

(57) ABSTRACT

Methods for interrogating aptamer-based electrochemical sensors using square wave voltammetry. A redox reporter is attached to each of the aptamers. Currents resulting from the interrogation are measured and used to determine an initial distribution of redox reporter above the surface of the aptamer-coated working electrode of the sensor. The measured currents are used to determine a location or a distribution of the redox-active species in relation to the surface, and in turn the amount of analyte about the electrode.

### FIELD OF THE INVENTION

The present invention relates generally to electrochemical sensors useful in determining the amount of an analyte in a fluid, including biological fluids such as blood and interstitial fluid. More particularly, the invention provides an electrochemical sensor that is operated by way of an improved method which provides for higher reliability analyte determination and extended sensor life.

### BACKGROUND TO THE INVENTION

Some classes of electrochemical sensors are selective and capable of the real-time, continuous detection of target analytes, as well as single point measurements, including agents which are exogenous (e.g., pharmaceutical compounds and toxins) and also those which are endogenous (e.g., metabolites, proteins, hormones, and the like).

Electrochemical sensors show significant promise in the field of human and animal health. In that context, a sensor may be embodied in the form of a microneedle-based patch applied to skin. The microneedle forms a working electrode which is inserted into the skin so as to contact the interstitial fluid. The tip of the microneedle functions as the sensor electrode, with the analyte recognition element (such as an aptamer) being associated with the tip. This arrangement provides a minimally invasive platform for real-time, continuous in vivo target analyte detection, which is sufficiently sensitive and selective to function in the complex matrix of the interstitial fluid.

An electrochemical sensor typically comprises a working electrode being coated in an analyte recognition element that undergoes a conformational change upon analyte binding. A redox reporter (such as methylene blue) may be covalently linked to the analyte recognition element. The conformational change in the analyte recognition element alters the accessibility of the redox reporter to the electrode surface, thereby producing an analyte-induced change in the level of electron transport between the redox reporter and the electrode. In some circumstances, binding of the analyte brings the redox reporter proximal to the electrode surface, thereby increasing the level of electron transport and in turn increasing current through the electrode. In other circumstances, binding moves the reporter to distal to the electrode surface resulting in the opposite effects. Regardless, binding of the analyte results in a detectable change in electrode current.

Electrochemical sensors are typically interrogated by the application of an electrical potential across the working electrode and a counter electrode, and then measuring current flow after the potential is removed or changed.

One method of sensor interrogation is square wave voltammetry, whereby an electrical potential is applied to the working electrode in the form of a square wave. Voltammograms (i.e., current versus voltage) are generated, and peak current through the working electrode is determined. Analyte amount is determined by way of an earlier generated calibration curve defining a relationship between analyte amount and peak current. Square wave voltammetry can be customized for a particular application to provide an increase (“signal-on”) or a decrease (“signal-off”) in peak current in the presence of target analyte. Signal drift and diminishing signal gain can be problems, which are addressed by taking measurements at two square wave frequencies which are then used to generate kinetic differential measurement (KDM) values. KDM values are calculated by subtracting the normalized peak currents measured at signal-on and signal-off frequencies, then dividing by their average. Averaged KDM values collected over a range of target concentrations to create a calibration curve, fitted using a Hill-Langmuir isotherm.

Another method used for interrogation of electrochemical sensors is cyclic voltammetry. In this method, the voltage across the working electrode and reference electrode is modulated between two values (V₁ and V₂) at a fixed rate. When the voltage reaches V₂ the scan is reversed, and the voltage is modulated back to V₁. The voltage is measured between the reference electrode and the working electrode, while the current is measured between the working electrode and the counter electrode. The obtained measurements are plotted as a voltammogram. As for square wave voltammetry, peak current is used to determine the amount of analyte about the working electrode.

A further prior art interrogation method is chronoamperometry, which has been shown to achieve drift-free and sub-second-resolved aptamer-based sensing. The difference in electron transfer rate between bound and unbound analyte can be measured as differences in current decay lifetimes. Such lifetimes can be related to the concentration of the target in the sample. Because chronoamperometric lifetimes are a function of the fractional population of bound vs unbound receptors, they are less sensitive to progressive changes of the sensor interface relative to total current amplitude, which depends on the total number of aptamers. A variation called Intermittent Pulse Amperometry, can be used to achieve millisecond-resolved measurements of analyte binding kinetics. The output of each periodic pulse produces one forward and one reverse chronoamperograms, which can be subtracted to produce a differential current that is directly proportional to the concentration of target analyte.

A further prior art interrogation method is electrochemical impedance spectroscopy (EIS). EIS is a technique found useful in the analysis of interfacial properties related to biorecognition events such as aptamer-target folding occurring due to target analyte binding. Biosensing with EIS is based on the change of electron transfer resistance using a redox probe couple such as [Fe(CN)₆]. Dynamic impedance measurements are typically performed at a single frequency, and a calibration curve used to convert impedance measurements into analyte amounts.

Prior art methods for analyte quantification using square wave voltammetry or chronoamperometry present a few challenges. For example, determination of KDM by square wave voltammetry is temperature dependent. The method requires normalization in the absence of target analyte, making it unsuitable for in situ use in a human to determine the amount of an endogenous analyte such as a hormone. Chronoamperometry is strongly subject to capacitive current and any potential shift in the reference electrode.

Sensor degradation is a further problem that may arise where a sensor is repeatedly interrogated, as is the case for continuous real-time monitoring of a target analyte.

Problems as to sensor accuracy, reproducibility, and the time needed to perform a measurement also present.

Where a sensor is configured as a portable device, including a wearable device, power consumption of the device electronics may limit the operating time due to battery capacity. It is therefore desirable to lower power consumption in such circumstances.

It is an aspect of the present invention to provide an improvement in the operation electrochemical sensors so as to ameliorate or overcome any one or more of the problems described herein. It is a further aspect of the present invention to provide a useful alternative to prior art methods of interrogating electrochemical sensors.

The discussion of documents, acts, materials, devices, articles, and the like, is included in this specification solely for the purpose of providing a context for the present invention. It is not suggested or represented that any or all of these matters formed part of the prior art base or were common general knowledge in the field relevant to the present invention as it existed before the priority date of each provisional claim of this application.

### SUMMARY OF THE INVENTION

In a first aspect, but not necessarily the broadest aspect, the present invention provides a method for determining an amount of an analyte in a fluid, the method comprising:
providing an electrochemical sensor working electrode, the working electrode having associated therewith a plurality of analyte recognition elements each of which has a redox-active species associated therewith;
applying a potential to the working electrode according to a periodic waveform; and
measuring current values resulting from the application of the potential.

In one embodiment of the first aspect, each of the plurality of analyte recognition elements is associated with a surface of the working electrode, and the measured current value(s) is/are used to determine a location or a distribution of the redox-active species in relation to the surface.

In one embodiment of the first aspect, the location or the distribution is an initial location or an initial distribution.

In one embodiment of the first aspect, wherein the location or the distribution of the redox-active species is used to determine the extent to which the redox-active species mobilize toward the surface of the working electrode, and in turn the amount of analyte recognized by the plurality of analyte recognition elements.

In one embodiment of the first aspect, the measured current value(s) is/are used to determine the extent to which the redox-active species mobilize toward the surface of the working electrode, and in turn the amount of analyte recognized by the plurality of analyte recognition elements.

In one embodiment of the first aspect, the measured current value(s) are used to generate one or more current-potential relationships.

In one embodiment of the first aspect, each of the one or more current-potential relationships is a differential current-potential relationship.

In one embodiment of the first aspect, the one or more current-potential relationships each provide a peak current, and the peak currents are used to calculate one or more f values, each f value being indicative of the fraction of the redox-active species that is at or proximal to a surface of the working electrode.

In one embodiment of the first aspect, the f value is calculated according to Equation (3).

In one embodiment of the first aspect, the periodic waveform has a substantially fixed frequency, the periodic waveform being superimposed on an underlying swept potential.

In one embodiment of the first aspect, the underlying swept potential has a staircase form.

In one embodiment of the first aspect, the periodic waveform has a duty cycle of about 50%.

In one embodiment of the first aspect, the periodic waveform is step-shaped so as to provide substantially instantaneous changes in potential.

In one embodiment of the first aspect, the periodic waveform is a substantially square waveform.

In one embodiment of the first aspect, the periodic waveform is applied for a plurality of cycles.

In one embodiment of the first aspect, the periodic waveform is applied for at least about 3, 4, 5, 10, 20, 30, 40, 50, 60, 70, 80, 90, 100, 1000, 10000, or 100000 cycles.

In one embodiment of the first aspect, the periodic waveform has a substantially fixed frequency.

In one embodiment of the first aspect, the substantially square waveform is applied according to square wave voltammetry method.

In one embodiment of the first aspect, the measured current value(s) are used to provide a time-current relationship.

In one embodiment of the first aspect, the time-current relationship defines a change in current over time.

In one embodiment of the first aspect, the change in current over time is a current transient.

In one embodiment of the first aspect, the current transient is dependent upon the rate of electron transport between the redox-active species and the surface of the working electrode.

In one embodiment of the first aspect, the rate of transport is dependent upon the accessibility of the redox-active species to the surface of the working electrode.

In one embodiment of the first aspect, the accessibility of the redox-active species to the surface of the electrode is dependent upon proximity of the reporter to the surface of the working electrode.

In one embodiment of the first aspect, the time-current relationship is used to determine a distribution of the redox-active species having regard to the proximity of the redox-active species to the surface of the working electrode.

In one embodiment of the first aspect, the method comprises measuring current at 2, 3, or more, different time points within a cycle of the periodic waveform.

In one embodiment of the first aspect, the method comprises measuring current at 2, 3, or more, different frequencies of the periodic waveform.

In one embodiment of the first aspect, the method comprises calculating one or more current ratios from the measured currents.

In one embodiment of the first aspect, the time-current relationship is processed to determine the analyte amount.

In one embodiment of the first aspect, the processing comprises determining a rate of current decay occurring after the application of the potential.

In one embodiment of the first aspect, the time-current relationship is used as input into a method for determining an analyte amount by a chronoamperometry method.

In one embodiment of the first aspect, the periodic waveform has a first frequency, and the method comprises:
applying the potential using the periodic waveform at the first frequency and measuring a current resulting therefrom at a time point late in the periodic wave cycle and also at one or two earlier time points.

In one embodiment of the first aspect, the time-current relationship is a current transient, or is used to determine a peak current.

In one embodiment of the first aspect, a waveform having only a single frequency is applied to the working electrode.

In one embodiment of the first aspect, each of the plurality of analyte recognition elements is an aptamer or a functional equivalent thereof having a binding specificity for the analyte.

In one embodiment of the first aspect, the redox-active species is linked to the analyte recognition element.

In a second aspect, the present invention provides an electrochemical sensor apparatus or system comprising:
a working electrode having associated therewith (i) an analyte recognition element and (ii) an associated redox-active species spatially constrained within a layer adjacent to the electrode surface; and
a microprocessor-based controller,
wherein the microprocessor-based controller to configured to perform the method of any embodiment of the first aspect.

In one embodiment of the second aspect, the microprocessor-based controller is in electrical connection with the working electrode, or in wired or wireless network connection with another microprocessor-based controller that is in electrical connection with the working electrode.

In one embodiment of the second aspect, the microprocessor controller is configured to access and execute program instructions to perform the method of any embodiment of the first aspect.

In one embodiment of the second aspect, the electrochemical sensor apparatus or system comprises a variable power supply in electrical connection with the working electrode, wherein the program instructions direct the power supply to apply a potential to the working electrode according to the method of any embodiment of the first aspect.

In one embodiment of the second aspect, the program instructions direct the application of the potential in a periodic waveform, or a substantially square waveform.

In one embodiment of the second aspect, the electrochemical sensor apparatus or system comprises current measuring circuitry configured to measure an electrical current through the working electrode.

In one embodiment of the second aspect, the electrochemical sensor apparatus comprises electronic memory in operable association with the microprocessor-based controller and the current measuring circuitry, the electronic memory configured to store one or more currents measured by the current measuring circuitry.

In one embodiment of the second aspect, the microprocessor-based controller is configured to process the one or more currents measured by the current measuring circuitry to provide an analyte amount.

In a third aspect, the present invention comprises a computer-readable medium comprising program instructions configured to execute the method of any embodiment of the first aspect.

### BRIEF DESCRIPTION OF THE FIGURES

FIG. 1 illustrates a preferred method of the invention whereby a square wave potential at three frequencies is applied to the working electrode of an aptamer-based electrochemical sensor, with the current output being measured and used to calculate an f value according to Equation (3) herein.

FIG. 2 is a graph showing implementation of the method of FIG. 1 depicting the f value response using peak currents at Fon|Fnr|Foff in place of i(t₁), i(t₂), and i(t₃), in the context of a working electrode having a vancomycin sensitive aptamer with a methylene blue redox reporter.

FIG. 3 is a graph showing implementation of the method of FIG. 1 depicting the response of an aptamer-based vancomycin-sensitive electrode to varying concentrations of vancomycin (2 μm, 10 μm, and 200 μm) over a period of 24 hours, using square wave voltammetry to interrogate, and Equation (3) to calculate f values.

FIG. 4 is an alternative to the method of FIG. 1, whereby a square wave potential at a single frequency is applied to the working electrode of an aptamer-based electrochemical sensor, with the current output being measured and used to calculate an f value according to Equation (3) herein.

With the exception of the graphs, the drawings are not prepared to any particular scale or dimension and are not presented as being a completely accurate presentation of the various embodiments.

### DETAILED DESCRIPTION OF THE INVENTION AND PREFERRED EMBODIMENTS THEREOF

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

Throughout the description and the claims of this specification the word “comprise” and variations of the word, such as “comprising” and “comprises” is not intended to exclude other additives, components, integers, or steps.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

Where the term “determine”, “determining” and “determined” are used, it is not intended that these terms be necessarily interpreted to mean any completely accurate determination, although that meaning is not excluded. The terms may be interpreted to include an estimation, an approximation or even an indication.

The term “aptamer” is intended to include DNA and RNA aptamers. A “functional equivalent” of an aptamer includes related species such as a xenonucleic acid (XNA) and a peptide nucleic acid (PNA).

In a first aspect, but not necessarily the broadest aspect, the present invention provides a method for determining an amount of an analyte in a fluid, the method comprising:

providing an electrochemical sensor working electrode, the working electrode having associated therewith a plurality of analyte recognition elements of which has a redox-active species associated therewith;

applying changes in potential to the working electrode according to a periodic waveform;

measuring current values resulting from the application of the potential; and

using the measured current to provide a time-current relationship.

It has been found that a workable or advantageous method of interrogation for an electrochemical sensor is provided where, in one embodiment, the interrogating potential is applied to the working electrode as a periodic waveform (such as a square waveform). The current resulting from the applied potential is considered as a function of time, and is used in a time-based methodology (as exploited in chronoamperometry, for example) to determine the amount of target analyte.

In some embodiments of the method, the potential applied to the working electrode of the apparatus as a periodic waveform (such as a square waveform) is changed from one that is sufficient to convert and maintain the redox species in one redox state, to another that is sufficient to substantially immediately change the redox state of any redox-active species sufficiently close to the electrode to allow electron transfer across the electrode/solution interface. Expressed in an alternative manner, the potential (applied as a periodic waveform, such as a square waveform) is changed to one such that the current flowing through the electrode surface as a result of the redox-active species changing redox state is controlled predominantly or substantially by mass transport of the redox-active species toward the electrode. As a result of the change in potential, net electrical current flows through the electrode surface, typically with the current changing in magnitude over time.

Without wishing to be limited by theory in any way, it is proposed that a redox-active reporter that is tethered to the working electrode surface (by an aptamer, for example), and therefore capable of restricted movement only within a layer adjacent the electrode, the redox-active species nevertheless substantially freely diffuses within the layer. Accordingly, movement of the redox-active species may be modelled with some accuracy (although not necessarily complete accuracy) on Fick's first and second laws of diffusion. Thus, even though tethered the redox-active species may nevertheless move from a region of high concentration to a region of low concentration, the magnitude of the flux being proportional to the differences in concentration, in a manner expected for a freely diffusible (i.e., untethered) species. Moreover, the concentration gradient of the redox-active species in the layer changes over time in a manner expected for a freely diffusible species.

Fick's first and second laws were applied to a system whereby a redox-active species is spatially constrained to be within a layer adjacent to the electrode surface, but able to freely diffuse within that layer. Accordingly, it was possible to model diffusion of the constrained redox species, and therefore model changes in current (termed “current transient”) due to movement of the redox-active species relative to the electrode surface given that electron transfer increases as the redox-active species is more proximal to the surface.

The resulting modelled current transient resulting from movement of the redox-active species revealed a dependence on (i) the surface area of the electrode, (ii) the thickness of the layer adjacent to the electrode, (iii) the diffusion coefficient of the redox species in the layer adjacent to the electrode, (iv) the overall concentration of the redox species in layer adjacent to the electrode and (v) the distribution of the redox species within the layer adjacent to the electrode at the time the potential step was applied. These revelations have been found to be of practical consequence in relation to methods for the interrogation of an electrochemical sensor apparatus, the determination of redox-active species distribution, and in turn determination of the amount of an analyte recognised by the analyte recognition element.

Further investigations were carried out demonstrating the practical relevance of single current measurements and current ratios determined in the period immediately after application of the potential change. It was surprisingly found that at longer times after the change in potential was applied, a ratio of two measured currents was strongly dependent upon the thickness of the layer adjacent to the electrode and the diffusion coefficient of the redox species but only weakly dependent upon the initial distribution of the redox species, whereas the ratio of a current at a shorter time after the potential step was applied, compared to a current at a longer time, was more strongly dependent upon the initial distribution of the redox species as well strongly dependent upon the thickness of the layer adjacent to the electrode and the redox species diffusion coefficient. In addition, both these current ratios were modelled to be insensitive to the overall concentration of the redox species and the electrode area.

These modelled behaviours suggested that the later current ratio may be useful in obtaining a combined measure of the redox species diffusion coefficient in combination with the thickness of the layer adjacent to the electrode, which could be applied to the earlier current ratio to obtain an estimated measure of the initial distribution of the redox species, where the estimate is largely insensitive to changes in the overall concentration of the redox species in the layer adjacent to the electrode, the thickness of the layer adjacent to the electrode and the diffusion coefficient of the redox species in the layer adjacent to the electrode and the area of the electrode.

The equation for current over time derived from the model is shown as Equation (1), below:

$$i ( t ) = \frac{4 {zFADC}_{0}}{l} {\sum }_{n = 0}^{∞} ( f ( 1 - \frac{4 {( - 1 )}^{n}}{( 2 n + 1 ) π} ) + \frac{2 {( - 1 )}^{n}}{( 2 n + 1 ) π} ) {e}^{- {( 2 n + 1 )}^{2} {π}^{2} Dt / 4 {l}^{2}} \qquad ( 1 )$$

where:
i(t) is the electrical current at time t,
z is the number of moles electrons transferred per mole of redox species either oxidised or reduced at the electrode surface,
F is Faraday's Constant,
A is the area of the electrode,
D is the diffusion coefficient of the redox species in the layer adjacent to the electrode,
C₀ is the overall concentration of the redox species in the layer adjacent to the electrode,
l is the thickness of the layer containing the redox species adjacent to the electrode, and
f is the fraction of the redox species that is close to or at the electrode surface when the potential step is applied.

Thus, f may be used to determine the distribution of the redox-active species when an initial potential is applied, and in the instant before the change in potential is applied. The distribution of the redox-active species may be used to determine the amount of analyte recognised by to the analyte recognition element of the sensor.

Note from Equation (1) that by taking the ratio of currents at two different times the electrode area and redox species concentration terms cancel out. In addition, at a sufficiently long time the exponential terms with n>0 will be small enough relative to the n=0 exponential term such that Equation (1) can be approximated by Equation (2), as follows:

$$i ( t ) ∼ \frac{4 zFAD {C}_{o}}{l} ( f ( 1 - \frac{4}{π} ) + \frac{2}{π} ) {e}^{- {π}^{2} Dt / 4 {l}^{2}} →︀ for \cdot t \cdot > \cdot {t}_{min} … →︀ \qquad ( 2 )$$

Accordingly, by taking the ratio of current at two different times where Equation (2) applies, only the exponential term does not cancel out, so that an estimate of D/l² may be obtained.

According to this method, the current at least three different times during the current transient may be determined: i(t₁), i(t₂), and i(t₃). Optionally, a current at a fourth time i(t₄) may be determined. Time t₁ is selected to be a short time after the change in potential step is applied, t₃ and optionally t₄ at longer times after the potential step is applied and t₂ to be between t₁ and t₃ or optionally to be between t₁ and t₄.

At least two ratios of currents may then be determined. In a preferred embodiment of the invention the ratios i(t₁)/i(t₃) and i(t₂)/i(t₃) are determined. In other embodiments of the invention, the ratios i(i₁)/i(t₄) and/or i(t₂)/i(t₄) are determined. In some embodiments of the invention, current ratios at additional times can be calculated and used to improve the method by providing additional estimates of the derived parameters.

In a further embodiment of the method, a second change in electrode potential is performed in the context of a periodic waveform (such as a square waveform), the second change occurring after the first change in electrode potential. In this embodiment, the electrode is, by the application of the periodic waveform, held at the potential resulting from the first change in potential and for a sufficient time to substantially electrochemically oxidise or reduce all the redox-active species present in the tethered layer. The electrode potential is then changed in the direction of the initial potential. For example, if the electrode potential was initially held at a value where the redox-active species was reduced, the second potential change would be in the direction of a stronger reducing potential. If the electrode potential was initially held at a value where the redox-active species was oxidised, the second step would be in the direction of a stronger oxidising potential. The current resulting from the second potential change can be used to obtain an estimate of the non-faradaic current flowing at the electrode, due to capacitive double-layer charging for example as well as any faradaic current from redox species that are not confined in the layer adjacent to the electrode, and subtracted from the current used to calculate the current ratios disclosed above, to improve the accuracy of the results.

The potentials achieved by first, second and third changes in potential may occur in the context of a periodic waveform, such as a square waveform, and may correlate respectively to the minimal potential value of the waveform, the maximum potential value of the waveform, and the minimal potential value of the square waveform respectively (each potential being added to an underlying swept potential).

The inputs required for the chronoamperometry method involving Equation (3) are currents corresponding to three different times after a step potential is applied (i(t₁), i(t₂), and i(t₃)), along with the values of t₁, t₂ and t₃.

It has been found that peak currents resulting from square wave voltammetry can provide the step potential, and therefore to produce f values according to Equation (3) that can be used in turn to determine the concentration of a target analyte. In square wave voltammetry the potential is slowly swept from one potential to another using a square wave slowly ascending or descending in potential over the sweep. If the potential is swept over the appropriate window, a peak in the current at a potential in the sweep is produced. The baseline current either side of the peak is subtracted from the current value at the peak to produce what is termed the “peak current” value. It is unexpected that peak currents measured in this way are useful in the context of a chronoamperometry approach to determining analyte concentration (including, for example, calculating f according to Equation (3)), as when using square wave voltammetry, the current flowing is entirely or nearly entirely electron transfer rate controlled; i.e., the current is limited by the potential applied to the electrode rather than the mass transport of the redox species to the electrode. It may be considered that this approach violates an assumption used to derive the equation for f as outlined herein.

The use of square wave voltammetry to generate the input values for the f equation is potentially useful, as subtracting the baseline current to calculate the square wave voltammetry peak current allows for correction of charging current contributions as well as background faradaic currents not related to the redox specie of interest. The times used when using square wave voltammetry to generate i(t₁), i(t₂), and i(t₃) are also typically longer than those required when using a prior art chronoamperometry approach, which potentially lessens the charging current contribution and makes the potential application and current measurement task easier and/or less expensive as slower speed electronic componentry may be used.

The present invention may be implemented by at least the following two different methods for obtaining the peak currents by way of square wave voltammetry for use in the calculation of f.

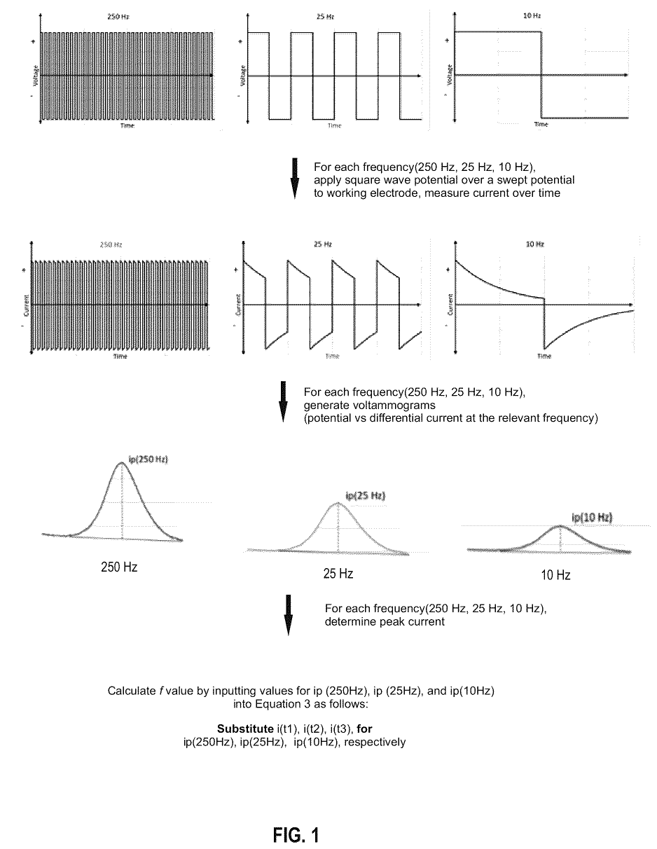

The first method requires that square wave voltammetry is performed at three different frequencies to obtain three peak currents, one peak current being obtained from each frequency sweep. This method is shown at FIG. 1, and detailed in Example 1 below.

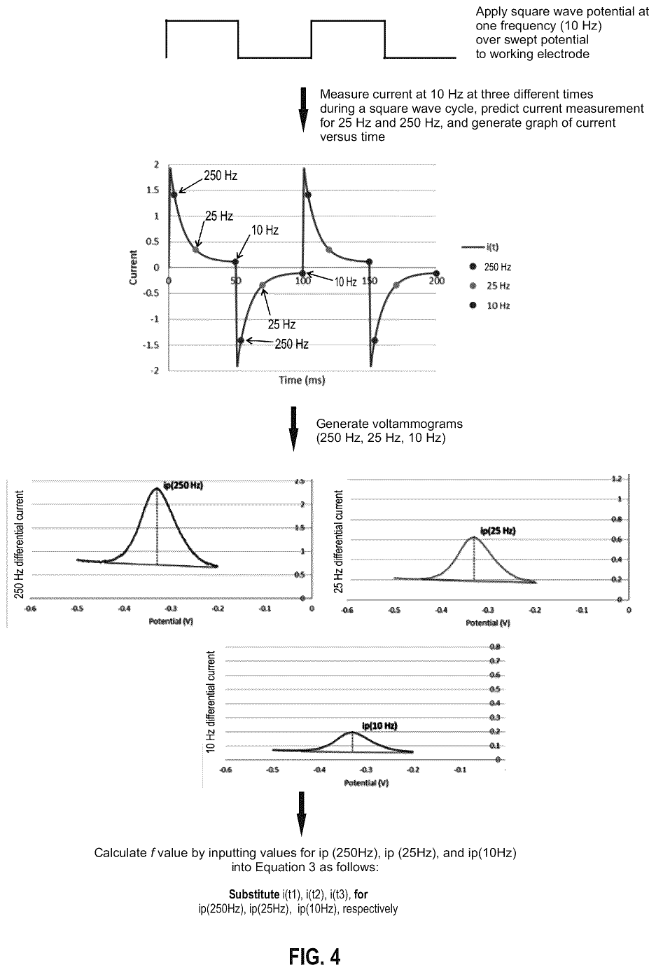

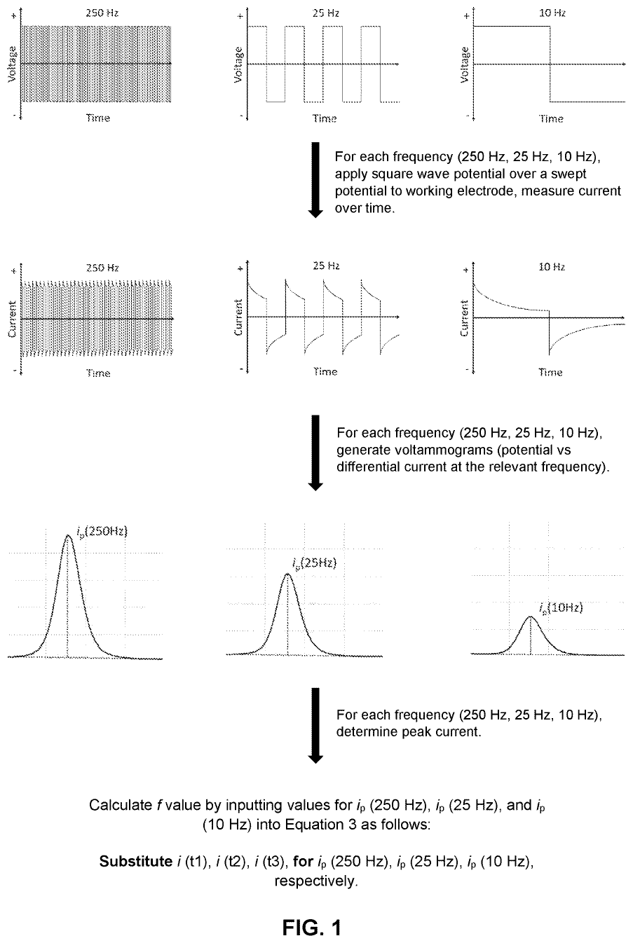

The second method is a modification of the first method, which requires a square wave voltammogram to be generated at only one frequency, typically the lowest frequency required to generate the data from input to the f equation. Instead of repeating a voltammogram sweep three times at different frequency, current is sampled at three different times during the square wave cycle at the lowest frequency of interest. This method is shown at FIG. 4, and detailed in Example 2 below.

The benefit of the second method over the first method is that it only requires a single frequency sweep instead of three, which saves time and battery life for a battery powered sensing device.

An advantage of some embodiments of the invention is that a method is provided which allows the distribution of the redox species to be estimated without specific knowledge of the electrode area, thickness of the tethered redox layer, overall amount of redox species, or number of electrons transferred per mole of redox species. In addition, or alternatively, application of the potential in the context of a periodic waveform (such as a square waveform) provides for a lower variation in the charging current. Furthermore, it allows for a range of potentials to be scanned so as to identify the optimal potential for a given redox-active species (such as methylene blue), and to accurately subtract this from non-redox-active species currents.

Another advantage of some embodiments of the invention is that a method is provided which is significantly faster than prior art interrogation methods, which may take seconds to minutes to execute. The presently described methods, in some embodiments, can be executed in tens of milliseconds. For example, the electrode is optionally held at an initial potential for a short time (of the order of tens of milliseconds to a second), and then the potential changed in a step-wise manner and held at a second potential for a time, typically up to tens of milliseconds. Because of the speed of execution and the fact that the redox species is tethered to the electrode, the stepping of potential can be repeated multiple times over a short period to obtain multiple estimates of the desired parameters that can be averaged or otherwise combined to reduce random variation in the results.

The present invention may be embodied in the form of an apparatus, or a system configured to facilitate execution of the methods described herein.

An apparatus of the present invention may be embodied in the form of a wearable device that is substantially self-contained, allowing measurements to be performed whilst the subject is undergoing normal activities and/or over a prolonged period of time. The wearable device may be a collar, a bracelet, a strap, an adhesive, or a patch. The wearable device may include transdermal microneedles, of which one of which functions as the working electrode of the sensor by contacting the interstitial fluid of the subject and detecting analytes therein.

The wearable device may further comprise a housing structure enclosing one or more other components, such as a processor-based microcontroller. The controller is configured to be in electrical communication with at least one electrode, and generally would include a power source, a data processing unit, electronic memory, and a wireless transmitter/receiver.

When embodied as a system, components may be distributed in different physical locations although still operate in an integrated manner. For example, software instructions may be stored and executed by a smart phone or other remote process in data communication with the microprocessor-based controller in a wearable device.

As will be understood, the methods described herein may be deployed in part or in whole through one or more microprocessors that execute computer software, program codes, and/or instructions on a processor. A microprocessor may be any kind of computational or processing device capable of executing program instructions, codes, binary instructions, and the like.

Any microprocessor may access a storage medium (such as electronic memory) through an interface that may store methods, codes, and instructions as described herein and elsewhere. The storage medium associated with the processor for storing methods, programs, codes, program instructions or other type of instructions capable of being executed.

The computer software, program codes, and/or instructions may be stored and/or accessed on computer readable media that may include: computer components, devices, and recording media that retain digital data used for computing for some interval of time; semiconductor storage known as random access memory (RAM); mass storage typically for more permanent storage, such as non-volatile memory such as read only memory (ROM).

The methods described herein may transform physical and/or or intangible items from one state to another. The methods and systems described herein may also transform data representing physical and/or intangible items from one state to another.

Software products may be created using a structured programming language such as C, an object oriented programming language such as C++, or any other high-level or low-level programming language (including assembly languages, hardware description languages, and database programming languages and technologies) that may be stored, compiled or interpreted to run on a microprocessor, as well as heterogeneous combinations of processors, processor architectures, or combinations of different hardware and software, or any other machine capable of executing program instructions.

Thus, in one aspect, any method may be embodied in computer executable code that, when executing on one or more microprocessors, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

The invention may be embodied in program instruction set executable on one or more microprocessors. Such instructions set may include any one or more of the following instruction types.

Data handling and memory operations, which may include an instruction to set a register to a fixed constant value, or copy data from a memory location to a register, or vice-versa, to store the contents of a register, result of a computation, or to retrieve stored data to perform a computation on it later, or to read and write data from hardware devices.

Arithmetic and logic operations, which may include an instruction to add, subtract, multiply, or divide the values of two registers, placing the result in a register, possibly setting one or more condition codes in a status register, to perform bitwise operations, e.g., taking the conjunction and disjunction of corresponding bits in a pair of registers, taking the negation of each bit in a register, or to compare two values in registers (for example, to determine if one is less, or if they are equal).

Control flow operations, which may include an instruction to branch to another location in the program and execute instructions there, conditionally branch to another location if a certain condition holds, indirectly branch to another location, or call another block of code, while saving the location of the next instruction as a point to return to.

Coprocessor instructions, which may include an instruction to load/store data to and from a coprocessor, or exchanging with CPU registers, or perform coprocessor operations.

A processor of a computer of the present system may include “complex” instructions in their instruction set. A single “complex” instruction does something that may take many instructions on other computers. Such instructions are typified by instructions that take multiple steps, control multiple functional units, or otherwise appear on a larger scale than the bulk of simple instructions implemented by the given processor. Some examples of “complex” instructions include: saving many registers on the stack at once, moving large blocks of memory, complicated integer, and floating-point arithmetic (sine, cosine, square root, etc.), SIMD instructions, a single instruction performing an operation on many values in parallel, performing an atomic test-and-set instruction or other read-modify-write atomic instruction, and instructions that perform ALU operations with an operand from memory rather than a register.

An instruction may be defined according to its parts. According to more traditional architectures, an instruction includes an opcode that specifies the operation to perform, such as add contents of memory to register—and zero or more operand specifiers, which may specify registers, memory locations, or literal data. The operand specifiers may have addressing modes determining their meaning or may be in fixed fields. In very long instruction word (VLIW) architectures, which include many microcode architectures, multiple simultaneous opcodes and operands are specified in a single instruction.

Some types of instruction sets do not have an opcode field (such as Transport Triggered Architectures (TTA) or the Forth virtual machine), only operand(s). Other unusual “0-operand” instruction sets lack any operand specifier fields, such as some stack machines including NOSC.

Conditional instructions often have a predicate field—several bits that encode the specific condition to cause the operation to be performed rather than not performed. For example, a conditional branch instruction is executed, and the branch taken, if the condition is true, so that execution proceeds to a different part of the program, and not executed, and the branch not taken, if the condition is false, so that execution continues sequentially. Some instruction sets also have conditional moves, so that the move is executed, and the data stored in the target location, if the condition is true, and not executed, and the target location not modified, if the condition is false. Similarly, IBM z/Architecture has a conditional store. Some instruction sets include a predicate field in every instruction; this is called branch predication.

The instructions constituting a program are rarely specified using their internal, numeric form (machine code); they may be specified using an assembly language or, more typically, may be generated from programming languages by compilers.

The present invention will now be more fully described by reference to the following non-limiting examples.

### Example 1: Square Wave Volammetry Method Using Three Different Frequencies

Reference is made to FIG. 1 detailing a preferred embodiment of the present method. The working electrode is excited with a potential applied as a square waveform. The potential is applied using square waveforms of three different frequencies (250 Hz, 25 Hz and 10 Hz). Relationships between differential current and potential are then derived for each frequency (shown as voltammograms in FIG. 1) to define a peak current (ip) in each case. The f value is calculated by inputting values for ip (250 Hz), ip (25 Hz), and ip (10 Hz) into Equation (3) (derived from Equation (1) and Equation (2), and shown below) by substituting i(t₁), i(t₂), i(t₃), at t₁, t₂, and t₃ in Equation (3) for ip (250 Hz), ip (25 Hz), ip (10 Hz) respectively.

$$f = - \frac{{\sum }_{n = 0}^{∞} ( \frac{2 {( - 1 )}^{n}}{( 2 n + 1 ) π} ) \cdot ( {r}_{1} {e}^{- {( 2 n + 1 )}^{2} {qt}_{3}} - {e}^{- {( 2 n + 1 )}^{2} {qt}_{1}} )}{{\sum }_{n = 0}^{∞} ( 1 - \frac{4 {( - 1 )}^{n}}{( 2 n + 1 ) π} ) \cdot ( {r}_{1} {e}^{- {( 2 n + 1 )}^{2} {qt}_{3}} - {e}^{- {( 2 n + 1 )}^{2} {qt}_{1}} )} →︀ \qquad ( 3 )$$

where r₁ is i(t₁)/i(t₃) and q is given by:

$$\text{?} \cdot \frac{{π}^{2} D}{4 {l}^{2}} = \frac{ln ( {r}_{2} )}{{t}_{3} - {t}_{2}} = q .$$

$$\text{?} \text{indicates text missing or illegible when filed}$$

It should be noted that the ratio i(t₂)/i(t₃) is present in the calculation for f, albeit not shown explicitly. To explain further, the variable r₂ is in fact:

$${r}_{2} = \frac{i ( {t}_{2} )}{i ( {t}_{3} )}$$

In Equation (3), terms of up to n=10, or even greater may be used to approximate the infinite series of exponentials

Multiple separate calculations for f may be carried out and averaged.

Considering now in more detail the method of FIG. 1, square wave voltammograms were generated using peak currents measured at three different frequencies: the signal-on frequency, the non-responsive frequency, and the signal-off frequency for three time points. Current was sampled just before the square wave flip, as is accepted practice in the art.

The peak current from the highest frequency scan (250 Hz) is used to obtain a value corresponding to i(t₁), the peak current from the middle frequency scan (25 Hz) is used to obtain a value corresponding to i(t₂), and the peak current from the lowest frequency scan (10 Hz) is used to obtain a value corresponding to i(t₃). The times, t₁, t₂ and t₃ that are needed to calculate the f values are given by 1/2F1, 1/2F2 and 1/2F3 respectively, where F1, F2 and F3 are the three frequencies used to generate the three square wave voltammograms.

According to this method the following equations are used to generate the input data for the equation to solve for f:

$$i ( {t}_{1} ) = i p ( F 1 ) and {t}_{1} = 1 / 2 F 1$$

$$i ( {t}_{2} ) = i p ( F 2 ) and {t}_{2} = 1 / 2 F 2$$

$$i ( {t}_{3} ) = i p ( F 3 ) and {t}_{3} = 1 / 2 F 3$$

where ip(F) denotes the square wave voltammetry peak current when using frequency F.

For an EAB sensor, three frequencies that have been found to be useful in this invention are where F1 is in a range whereby the peak current responds in one direction (increasing) when the target analyte is present in the solution; F3 is in a range where the peak current responds in the opposite direction (decreasing) when the target analyte is present in the solution; and F2 (which is between F1 and F3), being in a range where the peak current is relatively insensitive to the presence or absence of the target analyte. The frequency where the peak current increases in the presence of the target analyte is often termed in the art the “signal-on” frequency. This can be either the higher or the lower frequency, depending on the aptamer used. The frequency where the peak current decreases with the presence of the target analyte is termed the “signal-off” frequency. This can be either the higher or the lower frequency, depending upon the aptamer used, but is always the opposite one to the signal-on frequency. The frequency that is relatively unresponsive to the presence of the target is termed the “non-responsive frequency” or the “minimally responsive frequency”. This frequency will be between the signal-on and the signal-off frequencies. The invention need not be limited to using these three frequencies, but a high, intermediate, and low frequency will generally be used. For example, the three frequencies can be empirically determined by trial and error using different frequencies and observing the efficacy of the f values obtained in determining the analyte concentration.

For the vancomycin sensitive aptamer used in this Example, the higher frequency is the signal-on frequency, and the lower frequency is the signal-off frequency. Examples of suitable frequency ranges for and EAB sensor constructed using this aptamer corresponding to the signal-on, signal-off and non-responsive frequencies are 100 to 300 Hz for F1, 20 to 50 Hz for F2 and 5 to 20 Hz for F3. Suitable potential sweep windows for this aptamer with methylene blue as the redox reporter are, for example −0.45 V to −0.15 V, where the sweep may be started at −0.45 V or −0.15 V.

These observations regarding frequency selection and potential sweep may be applied also to the method performed using a single frequency, as detailed in Example 2.

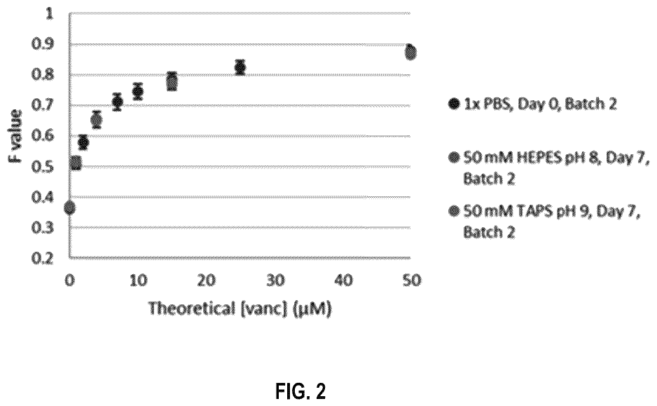

Reference is made to FIG. 2 showing correlation of the f value calculated as described above with vancomycin concentration. A direct correlation that appeared to fit a Langmuir isotherm is evident irrespective of the vehicle (i.e., PBS at neutral pH versus HEPES at pH 8 versus TAPS at pH 9). Furthermore, correlation was found to hold both before and after the sensors were stored for 7 days.

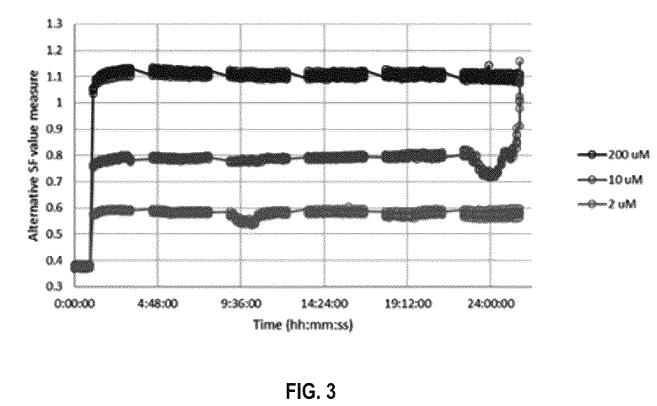

Reference is made to FIG. 3 showing the result of an experiment to quantify drift in f value over a 26-hour period, and over a range of vancomycin concentrations. It will be readily appreciated that for each concentration, little drift in the f value as determined by the method above was noted. The present methods may therefore be useful for interrogating an electrode disposed in situ in a human for continuous real-time monitoring of an analyte over an extended period of time.

### Example 2: Square Wave Volammetry Method Using a Single Frequency

Reference is made to FIG. 4, outlining a method for determining f value from peak current values. The difference in this method compared to that of Example 1 lies in how the i(t₁), i(t₂), and i(t₃) currents are generated. According to this method, a single square wave voltammogram is performed at the F3 frequency. This results in current transients as seen in the upper graph in FIG. 4. Instead of running separate square wave voltammetry at higher frequencies, additional current samples are taken from both the forward and reverse portions of the applied square wave potential. Using the example shown in FIG. 4, a voltammogram is constructed using the difference in current sampled at t₁ seconds after the potential is stepped in the forward and reverse directions to obtain a peak current corresponding to i(t₁), a second voltammogram is constructed using the difference in current sampled at t₂ seconds after the potential is stepped in the forward and reverse directions to obtain a peak current corresponding to i(t₂) and a third voltammogram is constructed using the difference in current sampled at t₃ seconds after the potential is stepped in the forward and reverse directions to obtain a peak current corresponding to i(t₃). The current sampling times chosen will typically correspond to 1/2F1, 1/2F2 and 1/2F3, where F1, F2 and F3 are as described above, but again may be empirically determined to obtain the most efficacious f values.

Those skilled in the art will appreciate that the invention described herein is susceptible to further variations and modifications other than those specifically described. It is understood that the invention comprises all such variations and modifications which fall within the spirit and scope of the present invention.

Accordingly, the spirit and scope of the present invention is not to be limited by the foregoing examples, but is to be understood in the broadest sense allowable by law.

1. A method for determining an amount of an analyte in a fluid, the method comprising:
providing an electrochemical sensor working electrode, the working electrode having associated therewith a plurality of analyte recognition elements each of which has a redox-active species associated therewith,
applying a potential to the working electrode according to a periodic waveform, and
measuring a current value resulting from the application of the potential at one or more time points within a cycle of the periodic waveform.

2. The method of claim 1, wherein each of the plurality of analyte recognition elements is associated with a surface of the working electrode, and the measured current value(s) is/are used to determine a location or a distribution of the redox-active species in relation to the surface.

3. The method of claim 2, wherein the location or the distribution is an initial location or an initial distribution.

4. The method of claim 2, wherein the location or the distribution of the redox-active species is used to determine the extent to which the redox-active species mobilize toward the surface of the working electrode, and in turn the amount of analyte recognized by the plurality of analyte recognition elements.

5. The method of claim 1, wherein the measured current value(s) is/are used to determine the extent to which the redox-active species mobilize toward the surface of the working electrode, and in turn the amount of analyte recognized by the plurality of analyte recognition elements

6. The method of claim 1, wherein the measured current value(s) are used to generate one or more current-potential relationships.

7. The method of claim 6, wherein each of the one or more current-potential relationships is a differential current-potential relationship.

8. The method of claim 6, wherein the one or more current-potential relationships each provide a peak current, and the peak currents are used to calculate one or more f values, each f value being indicative of the fraction of the redox-active species that is at or proximal to a surface of the working electrode.

9. The method of claim 8, wherein the f value is calculated according to Equation (3).

10. The method of claim 1, wherein the periodic waveform has a substantially fixed frequency, the periodic waveform being superimposed on an underlying swept potential.

11. (canceled)

12. (canceled)

13. (canceled)

14. (canceled)

15. (canceled)

16. (canceled)

17. (canceled)

18. The method of claim 1, wherein the periodic waveform is applied according to a square wave voltammetry method.

19. The method of claim 1, wherein the measured current value(s) are used to provide a time-current relationship

20. (canceled)

21. The method of claim 19, wherein the change in current over time is a current transient dependent upon the rate of electron transport between the redox-active species and a surface of the working electrode.

22. (canceled)

23. The method of claim 21, wherein the rate of transport is dependent upon the accessibility of the redox-active species to the surface of the working electrode.

24. The method of claim 23, wherein the accessibility of the redox-active species to the surface of the working electrode is dependent upon proximity of the reporter to the surface of the working electrode.

25. The method of claim 19, wherein the time-current relationship is used to determine a distribution of the redox-active species having regard to the proximity of the redox-active species to the surface of the working electrode.

26. The method of claim 1, comprising measuring current at 2, 3 or more, different time points within a cycle of the periodic waveform, or measuring current at 2, 3 or more, different frequencies of the periodic waveform.

27. (canceled)

28. (canceled)

29. (canceled)

30. (canceled)

31. (canceled)

32. The method of claim 1, wherein the periodic waveform has a first frequency, and the method comprises:
applying the potential using the periodic waveform at the first frequency and measuring a current resulting therefrom at a time point late in the periodic wave cycle and also at one or two earlier time points.

33. (canceled)

34. (canceled)

35. The method of claim 1, wherein each of the plurality of analyte recognition elements is an aptamer or a functional equivalent thereof having a recognition specificity for the analyte.

36. (canceled)

37. An electrochemical sensor apparatus or system comprising:
a working electrode having associated therewith (i) an analyte recognition element and (ii) an associated redox-active species spatially constrained within a layer adjacent to the electrode surface; and
a microprocessor-based controller,
wherein the microprocessor-based controller is configured to perform a method for determining an amount of an analyte in a fluid about the working electrode, the method comprising:
applying a potential to the working electrode according to a periodic waveform, and
measuring a current value resulting from the application of the potential at one or more time points within a cycle of the periodic waveform,
wherein the analyte recognition element is associated with a surface of the working electrode, and the measured current value(s) is/are used to determine a location or a distribution of the redox-active species in relation to the surface.

38. (canceled)

39. (canceled)

40. (canceled)

41. (canceled)

42. (canceled)

43. (canceled)

44. (canceled)

45. (canceled)
